(19) United States
(12) Patent Application Publication
(10) Pub. No.: US 2023/0092674 A1
(43) Pub. Date: Mar. 23, 2023

(54) Application of microbiota-derived plasmalogens to Treatment of Colon Cancer

(71) Applicant: Jiangnan University, Wuxi (CN)

(72) Inventors: Yuanfa LIU, Wuxi (CN); Yanjun LIU, Wuxi (CN); Yongjiang XU, Wuxi (CN)

(21) Appl. No.: 17/990,976

(22) Filed: Nov. 21, 2022

Related U.S. Application Data
(63) Continuation of application No. PCT/CN2022/091105, filed on May 6, 2022.

(30) Foreign Application Priority Data
May 11, 2021 (CN) 2021105096975

(51) Int. Cl. A61K 31/685; A61K 35/745; A61K 35/742; A61K 35/744; A61P 35/00; C12N 1/20; A23L 33/12

(52) U.S. Cl. CPC A61K 31/685; A61K 35/745; A61K 35/742; A61K 35/744; A61P 35/00; C12N 1/20; A23L 33/12; A23V 2002/00

(57) ABSTRACT

The present disclosure discloses application of a microbiota-derived plasmalogens to treatment of colon cancer, and belongs to the technical field of biomedicine and microorganisms. The microbiota-derived plasmalogens provided in the present disclosure is extracted from an anaerobic microorganism including any one or more of Lactobacillaceae, Bifidobacterium, Clostridium butyricum, and Peptostreptococcus Kluyver and van Niel after fermentation. The microbiota-derived plasmalogens can reduce expressions of colon cancer-associated cytokines at the molecular level, inhibit the proliferation of colon cancer cells, and reduce the number and size of colon adenomas in patients with colon cancer. The microbiota-derived plasmalogens can be used as an effective nutrition strategy for preventing and treating the colon cancer, and a theoretical support is provided for efficient utilization of the enteric microbiota-derived plasmalogens.

### TECHNICAL FIELD

The present disclosure relates to application of a microbiota-derived plasmalogens to treatment of colon cancer, and belongs to the technical field of biomedicine and microorganisms.

### BACKGROUND

As a genetic disease of colon tissues, colon cancer is induced by mutations of specific genes. Due to these mutations, a cell can have more advantages than nearby cells. When enough mutations of some genes occur, the cells may undergo cancerization and be divided uncontrollably to form tumors. At present, the colon cancer has become the second most common cancer cause of death all over the world. Risk factors include family histories, inflammatory bowel diseases, diabetes, low-fiber and high-fat diets, radiation therapy for cancer, hereditary colon cancer syndromes and the like. Currently, methods for prevention and relief of the colon cancer are still limited, and the use of less toxic and more natural methods in combination with current treatment methods to treat colon cancer more successfully is the research and industry trend at present.

As a class of glycerophospholipids, plasmalogens contains a vinyl-ether linked alkyl chain at the sn-1 position, and has been widely found in animal tissues and anaerobic microorganisms. It is worth noting that the plasmalogens were enriched in colon cells, and accounts for about 35% of total phospholipids. When the plasmalogens are lack in human cells, a variety of inflammatory and neurological diseases such as chronic obstructive pulmonary disease can be caused.

It has also been proven that exogenous plasmalogens have various biological activities, including improving oxidative stress levels and ameliorating neuroinflammation. However, limited studies has been made to the colon cancer. As a strategy for ameliorating the colon cancer, microbiota-derived plasmalogens needs to be studied urgently, which has great potential for protecting the overall health of patients.

### SUMMARY

An objective of the present disclosure is to provide a method for relieving colon cancer. The problem of limitations in treatment of colon cancer is solved. The present disclosure provides application of a microbiota-derived plasmalogens to preparation of a drug for treatment of colon cancer. The microbiota-derived plasmalogens is derived from an anaerobic microorganism.

In an embodiment, the application includes, but is not limited to, the use of the microbiota-derived plasmalogens as an active ingredient in a subject with or at a risk of colon cancer to relieve or treat symptoms of colon cancer disease.

In an embodiment, the microbiota-derived plasmalogens is derived from an anaerobic microorganism; and the microbiota-derived plasmalogens includes the following components: choline plasmalogen, ethanolamine plasmalogen, serine plasmalogen, phosphatidylglycerol plasmalogen, and phosphatidic acid plasmalogen.

In an embodiment, as an active ingredient in the drug for treatment of colon cancer, the microbiota-derived plasmalogens has a minimum dose of 5 mg/kg·BW.

In an embodiment, the anaerobic microorganism is a mixture of any one or more of Lactobacillaceae, Bifidobacterium, Clostridium butyricum, and Peptostreptococcus Kluyver and van Niel.

In an embodiment, the anaerobic microorganism includes Bifidobacterium longum and the C. butyricum.

In an embodiment, the microbiota-derived plasmalogens includes the following components: 158.3 mg/g of the choline plasmalogen (PlsCho), 241.8 mg/g of the ethanolamine plasmalogen (PlsEtn), 11.0 mg/g of the serine plasmalogen (PlsSer), 367.1 mg/g of the phosphatidylglycerol plasmalogen (PlsGly), and 181.0 mg/g of the phosphatidic acid plasmalogen (PlsOH).

In an embodiment, the microbiota-derived plasmalogens has a purity of greater than 90%.

In an embodiment, the drug is prepared in any one of the following dosage forms: a tablet, a capsule, a granule, a powder, and a liquid preparation.

In an embodiment, the treatment of colon cancer includes reducing expressions of colon cancer-associated cytokines IL-6, TNFα, and COX2, reducing the number and size of colon adenomas, or inhibiting the proliferation of colon cancer cells.

The present disclosure further provides application of a microbiota-derived plasmalogens to preparation of a health care product for relieving symptoms of colon cancer. The microbiota-derived plasmalogens has a minimum dose of 5 mg/kg·BW.

The present disclosure further provides application of a microbiota-derived plasmalogens to preparation of food for relieving symptoms of colon cancer. The microbiota-derived plasmalogens has a minimum dose of 5 mg/kg·BW.

In an embodiment, the microbiota-derived plasmalogens is an active ingredient of an enteral nutrition preparation, a dietary supplement, a veterinary drug, or a feed additive.

The present disclosure further provides a drug for treatment of colon cancer. The drug includes a microbiota-derived plasmalogens. The microbiota-derived plasmalogens is derived from an anaerobic microorganism. The microbiota-derived plasmalogens includes the following components: choline plasmalogen, ethanolamine plasmalogen, serine plasmalogen, phosphatidylglycerol plasmalogen, and phosphatidic acid plasmalogen.

In an embodiment, the microbiota-derived plasmalogens includes the following components: 158.3 mg/g of the choline plasmalogen, 241.8 mg/g of the ethanolamine plasmalogen, 11.0 mg/g of the serine plasmalogen, 367.1 mg/g of the phosphatidylglycerol plasmalogen, and 181.0 mg/g of the phosphatidic acid plasmalogen.

In an embodiment, the microbiota-derived plasmalogens is obtained by culturing the anaerobic microorganism in an MRS culture medium.

In an embodiment, the microbiota-derived plasmalogens is prepared in the following steps:

(1) separately culturing B. longum and C. butyricum in an MRS liquid culture medium for at least 24 hours, and collecting bacteria;

(2) freeze-drying and breaking bacterial cells obtained in step (1);

(3) conducting sub-critical extraction on a broken cell powder obtained in step (2) to obtain a phospholipid complex containing a microbiota-derived plasmalogens;

(4) conducting enzymolysis on the phospholipid complex obtained in step (3) with a phospholipase A1; and

(5) extracting an enzymatic phospholipid obtained in step (4) to obtain a microbiota-derived plasmalogens with a purity of equal to or greater than 90%.

Beneficial effects: The microbiota-derived plasmalogens can reduce expressions of colon cancer-associated cytokines at the molecular level, inhibit the proliferation of colon cancer cells, and reduce the number and size of colon adenomas in patients with colon cancer. The microbiota-derived plasmalogens can be used as an effective nutrition strategy for treating and relieving the colon cancer, and a theoretical support is provided for efficient utilization of the enteric microbiota-derived plasmalogens.

### BRIEF DESCRIPTION OF FIGURES

FIG. 1 shows the influence of a microbiota-derived plasmalogens on the number and tumor size of colon adenomas in an embodiment of the present disclosure. The left figure shows the influence of a microbiota-derived plasmalogens on the size of colon adenomas, the abscissa refers to groups, and the ordinate refers to the tumor size. The right figure shows the influence of a microbiota-derived plasmalogens on the number of colon adenomas, the abscissa refers to groups, and the ordinate refers to the tumor number. ** indicates that P is less than 0.01.

FIG. 2 shows the influence of a microbiota-derived plasmalogens on expression quantities of colon cancer-associated inflammatory factors in the colon of mice with colon cancer in an embodiment of the present disclosure. The expression quantities of the inflammatory factors include mRNA expression quantities of IL-6, mRNA expression quantities of TNF-α, and mRNA expression quantities of Cox-2. The abscissa refers to groups, and the ordinate refers to the expression quantities. ** indicates that P is less than 0.01.

FIG. 3 shows the inhibition of a microbiota-derived plasmalogens on growth of a colon cancer cell line in an embodiment of the present disclosure. The abscissa refers to groups, and the ordinate refers to the cell survival rate. ** indicates that P is less than 0.01.

### DETAILED DESCRIPTION

A human colon cancer cell strain HT-29 was derived from an ATCC cell bank. Mice were purchased from Charles River Biological Co., LTD.

B. longum, with a deposit number of CICC 24632, was purchased from China Center of Industrial Culture Collection. C. butyricum, with an article number of 246167, was purchased from Mingzhou Biological Co., LTD.

### Example 1 Preparation of a Microbiota-Derived Plasmalogens

Microbiota-derived plasmalogens were produced from a strain of an anaerobic microorganism, including a combination of one or more strains of Lactobacillaceae, Bifidobacterium, C. butyricum, P. Kluyver and van Niel, Veillonella, and other positive plasmalogen microorganisms (that is, plasmalogen-containing microorganisms).

In an embodiment, the microbiota-derived plasmalogens was prepared in the following steps.

1) B. longum and C. butyricum were separately cultured in an MRS liquid culture medium for 24 hours at an inoculation amount of 0.1%. Culture solutions of the B. longum and C. butyricum were separately collected and centrifuged at 3,500 rpm/min, and bacteria were collected. Compositions of plasmalogens of the two microorganisms are shown in Table 1. A liquid chromatography-mass spectrometry technology was used as a method for detecting the compositions. That is, lipids were extracted from samples with isopropanol. An ACQUITY UPLC liquid chromatography system and a TripleTOF 5600 mass spectrometry system were used for determining the compositions of the lipids. A BEH C18 column (50×2.1 mm; 1.7 μm) was used for separation at a temperature of 45° C. and a flow rate of 0.3 mL/min. Parameters were as follows: collision energy, 40 eV; impact energy diffusion, 15 eV; cycle time, 1,300 ms; and temperature, 300° C. (−). With the peak area as the proportional unit of compositions, contents of the compositions in products obtained were calculated.

TABLE 1
Plasmalogens of B. longum and C. butyricum

| | | Bifidobacterium | Clostridium |
|---|---|---|---|
| | Molecular compositions of plasmalogens | longum | butyricum |
| Phosphatidic | PlsOH O—32:0 \| | 15614 ± 2154 | 14567 ± 1278 |
| acid | PlsOH O—17:0_15:0 \| [M—H]— | | |
| plasmalogen | PlsOH O—31:1 \| | 24723 ± 1586 | 13455 ± 786 |
| | PlsOH O—15:1_16:0 \| [M—H]— | | |
| | PlsOH O—33:2 \| | 2373 ± 578 | 1734 ± 72 |
| | PlsOH O—17:1_16:1 \| [M—H]— | | |
| Choline | PlsOH O—31:1 \| PlsCho O— | 54832 ± 1506 | 47084 ± 1276 |
| plasmalogen | 15:1_16:0 \| [M + CH3COO]— | | |
| | PlsCho O—37:1 \| PlsCho O— | 4269 ± 59 | 3818 ± 138 |
| | 19:0_18:1 \| [M + CH3COO]— | | |
| Ethanolamine | PlsEtn O—31:1 \| | 12820 ± 452 | 6224 ± 2054 |
| plasmalogen | PlsEtn O—15:1_16:0 \| [M—H]— | | |
| | PlsEtn O—33:2 \| | 5397 ± 168 | 8474 ± 1073 |
| | PlsEtn O—17:1_16:1 \| [M—H]— | | |
| | PlsGly O—29:1 \| | 28364 ± 1059 | 15348 ± 1258 |
| | PlsGly O—14:1_15:0 \| [M—H]— | | |
| Phosphatidyl- | PlsGly O—31:1 \| | 107267 ± 2405 | 125883 ± 2875 |
| glycerol | PlsGly O—15:1_16:0 \| [M—H]— | | |
| plasmalogen | PlsGly O—31:1 \| | 109214 ± 1538 | 57854 ± 1052 |
| | PlsGly O—16:1_15:0 \| [M—H]— | | |
| | PlsGly O—32:0 \| | 17363 ± 458 | 24411 ± 1057 |
| | PlsGly O—17:0_15:0 \| [M—H]— | | |
| | PlsGly O—33:1 \| | 40615 ± 896 | 27167 ± 1078 |
| | PlsGly O—17:1_16:0 \| [M—H]— | | |
| | PlsGly O—33:2 \| | 95079 ± 786 | 63704 ± 4137 |
| | PlsGly O—17:1_16:1 \| [M—H]— | | |
| Serine | PlsSer O—33:2 \| | 15069 ± 264 | 12154 ± 846 |
| plasmalogen | PlsSer O—17:1_16:1 \| [M—H]— | | |
| L-α- | LysoPlsEtn O—15:1 \| [M—H]— | 22481 ± 457 | 18580 ± 305 |
| lysophosphatid | LysoPlsEtn O—17:1 \| [M—H]— | 4623 ± 467 | 3162 ± 108 |
| ylethanolamine | LysoPlsEtn O—19:0 \| [M—H]— | 48784 ± 998 | 47240 ± 2475 |
| plasmalogen | LysoPlsGly O—17:1 \| [M-H]— | 9779 ± 705 | 9772 ± 129 |

2) The bacteria of the B. longum and C. butyricum were mixed at a ratio of 1:1, freeze-dried under vacuum, and then pulverized to obtain a powder with a lipid content of 15.9%.

3) Sub-critical extraction was conducted on the powder to obtain a phospholipid complex. Specific operation parameters were as follows: solution-material ratio, 1:1 to 1.5:1; operation pressure of an extraction tank, 0.3-0.8 MPa; extraction temperature, 30-50° C.; extraction time, 30-60 minutes; and temperature of a separation tank, 50-70° C.

4) The phospholipid complex obtained in step 3) was dissolved in n-hexane, and then added into a heat collecting agitator (at 200 r/min) for a reaction for 20 hours by controlling the substrate concentration at 2 g/mL, the enzyme addition amount (phospholipase A1, Lectiase Ultra) at 4%, the water addition amount at 10%, and the reaction temperature at 45° C. A reaction solution was subjected to centrifugation (at 4,000 r/min) and rotary evaporation for 30 minutes to obtain an enzymatic phospholipid.

5) 50 g of the enzymatic phospholipid was packed and sealed, and then the temperature and pressure were increased to meet the following predetermined extraction conditions: extraction temperature, 50° C.; extraction pressure, 35 MPa; ethanol amount, 3 kg/h; and extraction time, 5 hours.

The collected plasmalogen is a plasmalogen mixture which contains a fatty acid at the sn-1 position linked to a glycerol skeleton by a vinyl ether bond, and contains odd or even chains of fatty acids. The plasmalogen mainly includes a mixture of one or more of choline plasmalogen (PlsCho), ethanolamine plasmalogen (PlsEtn), serine plasmalogen (PlsSer), phosphatidylglycerol plasmalogen (PlsGly), and phosphatidic acid plasmalogen (PlsOH).

Compositions of the collected microbiota-derived plasmalogens with high purity are shown in Table 2. The microbiota-derived plasmalogens has a purity of 95.84%, and is used for functional evaluation.

TABLE 2
Analysis of compositions of microbiota-derived plasmalogens

| | PlsOH | PlsCho | PlsGly | PlsEtn | PlsSer |
|---|---|---|---|---|---|
| Compositions | 181.0 mg/g | 158.3 mg/g | 367.1 mg/g | 241.8 mg/g | 11.0 mg/g |
| of | | | | | |
| plasmalogen | | | | | |

### Example 2 Application of a Microbiota-Derived Plasmalogens to Relief/Treatment of Colon Cancer

1. Modeling

45 6-week-old C57BL/6J male mice with an individual weight of 20-22 g were evenly divided into three groups including a solvent control group, a model group, and a microbiota-derived plasmalogens group.

In the microbiota-derived plasmalogens group, model animals with colon cancer were intraperitoneally injected with azoxymethane (AOM, 10 mg/kg·BW) once. A week later, the model animals continuously drank a sodium sulfate solution containing 3% of glucan for 7 days, and then continuously drank normal drinking water for 7 days. With 14 days as a cycle, the operations above were repeated for a total of 3 cycles.

In the solvent control group, the mice were intraperitoneally injected with normal saline in the same volume as the AOM on the first day, and then drank normal drinking water for the same period as the sample group.

2. Treatment

In the microbiota-derived plasmalogens group, the mice were orally given the microbiota-derived plasmalogens obtained in step 1) by gavage (1.0 mg/kg·BW) every day for 6 weeks.

In the solvent control group, the mice were given 1.0 mg/kg·BW of normal saline every day for 6 weeks. After the intervention was completed, the mice were killed with CO₂ until the heart was ischemic. Then, spleens of the animals were taken and weighed, and organ coefficients were recorded. The whole colon and rectum from the anus to the tail end of the cecum were taken, vertically opened along the colic band, and rinsed with normal saline. Then, lesions in the intestinal tract were observed, and formed tumors were counted.

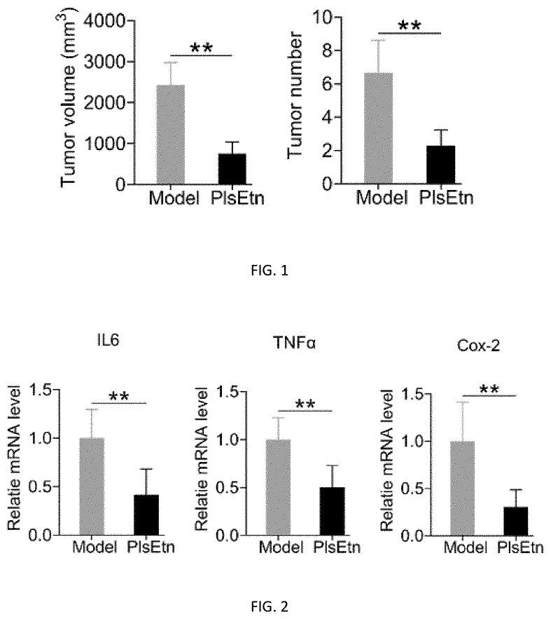

According to results, the AOM/DSS model induced mice have different degrees of thickened fold disorders in the intestinal mucosa, and the growth of colorectal tumors with different numbers and sizes is observed, indicating that the modeling is successful. As shown in FIG. 1, compared with the solvent control group, the number and size of colon tumors in the microbiota-derived plasmalogens group are significantly reduced by 72.4% and 61.9%, respectively (P<0.01; P<0.01).

According to results as shown in FIG. 2, after detection of mRNA expression levels of various cellular inflammatory factors in colon tissues by QtPCR, it is found that the mRNA expression levels of colonic cytokines IL-6 (P<0.01), TNFα (P<0.01), and COX2 (P<0.01) in the microbiota-derived plasmalogens group are significantly reduced.

3. HT-29 Colon Cancer Cell Experiment

A human colon cancer cell strain HT-29 was allowed to grow in a DMEM culture medium containing 10% of fetal bovine serum, and then cultured in a cell incubator at 37° C. and a saturated humidity of 5% CO₂. A culture solution was changed every 2-3 days, and cells in the logarithmic growth period were taken for an experiment. A human colon cancer cell strain HT-29 was cultured in vitro, and cells were interfered with the microbiota-derived plasmalogens obtained in step 1). That is, the cells were inoculated into a 96-well plate with about 5×10³ cells in each well, and cultured in a constant-temperature incubator. After the cells were attached to the wall, the microbiota-derived plasmalogens with a concentration of 100 μmol/L (dissolved in DMSO) was added. In a control group, DMSO was added in the same volume (5 μL). After the cells were cultured for 24 hours, the proliferation of the cells was detected by an MTT method.

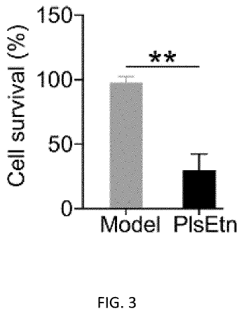

According to results as shown in FIG. 3, the proliferation of the colon cancer cells HT-29 is inhibited by the microbiota-derived plasmalogens, and reduced by 67.6% (P<0.01).

According to the above results, it is shown that the microbiota-derived plasmalogens has the effects of reliving and treating colon cancer.

Although the present disclosure has been disclosed as above in preferred embodiments, the embodiments are not intended to limit the present disclosure. Various changes and modifications can be made by any person familiar with the art without departing from the spirit and scope of the present disclosure. Therefore, the protection scope of the present disclosure should be defined by the claims.

What is claimed is:

1. Application of a microbiota-derived plasmalogens, comprising preparing a drug using microbiota-derived plasmalogens for treatment of colon cancer, wherein the microbiota-derived plasmalogens is derived from an anaerobic microorganism; and the microbiota-derived plasmalogens comprises the following components: choline plasmalogen, ethanolamine plasmalogen, serine plasmalogen, phosphatidylglycerol plasmalogen, and phosphatidic acid plasmalogen.

2. The application according to claim 1, wherein the microbiota-derived plasmalogens comprises the following components: 158.3 mg/g of the choline plasmalogen, 241.8 mg/g of the ethanolamine plasmalogen, 11.0 mg/g of the serine plasmalogen, 367.1 mg/g of the phosphatidylglycerol plasmalogen, and 181.0 mg/g of the phosphatidic acid plasmalogen.

3. The application according to claim 2, wherein the microbiota-derived plasmalogens has a minimum intake dose of 5 mg/kg·BW.

4. The application according to claim 1, wherein the anaerobic microorganism is a mixture of any one or more of Lactobacillaceae, Bifidobacterium, Clostridium butyricum (C. butyricum), and Peptostreptococcus Kluyver and van Niel.

5. The application according to claim 4, wherein the anaerobic microorganism comprises Bifidobacterium longum and the C. butyricum.

6. The application according to claim 1, wherein the microbiota-derived plasmalogens has a purity of greater than 90%.

7. The application according to claim 1, wherein the drug is prepared in any one of the following dosage forms: a tablet, a capsule, a granule, a powder, and a liquid preparation.

8. The application according to claim 1, wherein the treatment of colon cancer comprises reducing expressions of colon cancer-associated cytokines IL-6, TNFα, and COX2, reducing the number and size of colon adenomas, or inhibiting the proliferation of colon cancer cells.

9. Application of a microbiota-derived plasmalogens to preparation of a health care product for relieving symptoms of colon cancer, wherein the microbiota-derived plasmalogens has a minimum dose of 5 mg/kg·BW; and the microbiota-derived plasmalogens comprises the following components: 158.3 mg/g of choline plasmalogen, 241.8 mg/g of ethanolamine plasmalogen, 11.0 mg/g of serine plasmalogen, 367.1 mg/g of phosphatidylglycerol plasmalogen, and 181.0 mg/g of phosphatidic acid plasmalogen.

10. The application of claim 9, wherein the health care product is a food.

11. A drug for treatment of colon cancer, wherein the drug comprises a microbiota-derived plasmalogens; the microbiota-derived plasmalogens is derived from an anaerobic microorganism; and the microbiota-derived plasmalogens comprises the following components: choline plasmalogen, ethanolamine plasmalogen, serine plasmalogen, phosphatidylglycerol plasmalogen, and phosphatidic acid plasmalogen.

12. The drug according to claim 11, wherein the microbiota-derived plasmalogens comprises the following components: 158.3 mg/g of the choline plasmalogen, 241.8 mg/g of the ethanolamine plasmalogen, 11.0 mg/g of the serine plasmalogen, 367.1 mg/g of the phosphatidylglycerol plasmalogen, and 181.0 mg/g of the phosphatidic acid plasmalogen.

13. The drug according to claim 11, wherein the drug is prepared in any one of the following dosage forms: a tablet, a capsule, a granule, a powder, and a liquid preparation.

14. The drug according to claim 11, wherein the microbiota-derived plasmalogens is obtained by culturing the anaerobic microorganism in an MRS culture medium.
